(19) United States
(12) Patent Application Publication
(10) Pub. No.: US 2011/0180436 A1
(43) Pub. Date: Jul. 28, 2011

(54) MAYO STAND TRAY AND COVER

(76) Inventors: Fabian von Posern, Solana Beach, CA (US); John Gamble, Solana Beach, CA (US)

(21) Appl. No.: 12/695,006

(22) Filed: Jan. 27, 2010

(51) Int. Cl. B65D 1/34; A61B 19/02

(52) U.S. Cl. 206/370; 206/559

(57) ABSTRACT

A mayo stand having a springy material positioned on the mayo stand tray. The springy material is of sufficient softness and thickness so that it is easily depressed downward by the finger and thumb force of a doctor or his assistant so that a surgical tool on the tray is easily graspable. In a preferred embodiment the springy material is cut from a ½ inch thick foam polyurethane sheet and is adhesively attached to a thin, flexible plastic bag. The thin, flexible plastic bag is clean and sterile and covers the mayo stand and the mayo stand tray. Preferably the flexible plastic bag is a single use item that is disposed of following the completion of the medical procedure.

The present invention relates to mayo stands and, in particular, to the mayo stand trays and mayo stand covers.

### BACKGROUND OF THE INVENTION

Mayo stands are commonly used in hospitals and doctors' offices during medical procedures. FIG. 1 shows a perspective view of prior art uncovered Mayo stand 1. Prior to utilization, a mayo stand cover is draped over the mayo stand. Then, surgical tools and instruments are placed on top of tray 6 of mayo stand 1 for access by the doctor performing the medical procedure.

FIG. 2 shows a perspective view of mayo stand 1 in the process of being covered by prior art mayo stand cover 2. Prior art mayo stand cover 2 is a single-use, disposable item. Mayo stand cover 2 includes thin flexible plastic bag 3. Flexible bag 3 is open at end 4 and closed at end 5 (FIG. 3). Very thin, heavy-duty absorbent paper sheet 2b is adhesively attached to the top of flexible plastic bag 3 as shown in FIG. 3. The purpose of the absorbent paper sheet 2b is to absorb blood or other fluids that become attached to the surgical tools during the medical procedure. In the example shown in FIGS. 2 and 3, plastic bag 3 is approximately 53 inch×23 inch and absorbent paper sheet 2b is approximately 25½ inch×21 inch.

To prepare mayo stand 1 for a medical procedure mayo stand cover 2 is slipped over mayo stand 1 (FIG. 2) so that it appears as shown in FIG. 5. Tray 6 shown in FIG. 1 has dimensions of approximately 19⅛ inch×12⅝ inch. Absorbent paper sheet 2b (FIG. 3) is situated so that it is covering the top of tray 6 of mayo stand 1. Thin absorbent cotton cloth 7 (FIG. 6) is then laid over the top of mayo stand cover 2 and tray 6. Medical tools 8 are then placed on top of cotton cloth material 7 as shown.

In the prior art, sterility and safety are paramount. For example, mayo stand cover 2 and cotton cloth material 7 are single use, disposable items and are thrown away after the medical procedure is completed. To even further ensure safety, cleanliness and sterility, doctors and their assistants are required to scrub their hands prior to all medical procedures. Furthermore, the doctors and their assistants are required to wear surgical gloves prior to the procedure.

Typically, a doctor performing a medical procedure will grab as needed a medical tool resting on the mayo stand with his hands. Or, a doctor will call for a specific tool and an assistant will grab the tool resting on the mayo stand. The assistant will then hand the tool to the doctor as requested.

The top of tray 6 is very hard and flat. Many of the tools are very small and flat. Consequently, doctors and their assistants frequently find it difficult to grasp the small tools with their fingers. Additionally, the doctors and assistants wear surgical gloves, which make it even more difficult to grab the small tools. Also, doctors with large hands have a further challenge trying to grab small, flat tools. If the medical procedure is long or the medical team is fatigued this can be a further complication. The danger is that the medical team can become frustrated, irritated and their concentration can become affected. Tools can be dropped, frustration levels can increase and valuable seconds can be lost. It is very important that all medical personal are performing at the best of their ability.

For example, FIG. 7 shows a side view of tray 6 covered by plastic sheet 3, absorbent paper sheet 2b and cloth 7. Tool 8 is positioned on the top of cloth 7 as shown. In FIG. 7 a doctor is beginning to attempt to pick up tool 8. In FIG. 8, the doctor has moved his fingers closer together and is touching tool 8. However the tool is small and flat, and the doctor is having difficulty grabbing the tool to pick it up.

Polyethylene foam is known. Polyethylene foam is a strong, resilient closed cell foam. It is commonly used for shock absorbing, vibration dampening, and loose fill. It's most common use is for cushioning products in packaging applications. Polyethylene is lightweight, flexible, cost-effective, and impervious to mildew, mold, rot, and bacteria. Soft polyethylene foam functions as a spring in that it will spring back to its original shape after being depressed and then released.

Polyurethane foam is also known. A polyurethane is any polymer consisting of a chain of organic units joined by urethane (carbamate) links. Polyurethane polymers are formed through step-growth polymerization by reacting a monomer containing at least two isocyanate functional groups with another monomer containing at least two hydroxyl (alcohol) groups in the presence of a catalyst. Soft polyurethane foam, like polyethylene foam, also functions in a spring-like fashion in that it will spring back to its original shape after being depressed and then released. For example, soft polyurethane foam is widely used for car seats due to its high resiliency and spring properties.

What is needed is a mayo stand tray in which it is easy to pick up small, flat surgical tools.

### SUMMARY OF THE INVENTION

The present invention provides a mayo stand having a springy material positioned on the mayo stand tray. The springy material is of sufficient softness and thickness so that it is easily depressed downward by the finger and thumb force of a doctor or his assistant so that a surgical tool on the tray is easily graspable. In a preferred embodiment the springy material is cut from a ½ inch thick foam polyurethane sheet and is adhesively attached to a thin, flexible plastic bag. The thin, flexible plastic bag is clean and sterile and covers the mayo stand and the mayo stand tray. Preferably the flexible plastic bag is a single use item that is disposed of following the completion of the medical procedure.

### BRIEF DESCRIPTION OF THE DRAWINGS

FIG. 1 shows a prior art mayo stand.

FIG. 2 shows a prior art mayo stand in the process of being covered by a mayo stand cover sheet.

FIGS. 3 and 4 show prior art mayo stand cover sheets.

FIG. 5 shows a prior art mayo stand covered by a mayo stand cover sheet.

FIG. 6 shows surgical tools positioned on a prior art mayo stand tray.

FIGS. 7 and 8 show a doctor trying to pick up a surgical tool from a prior art mayo stand tray.

FIG. 9 shows a preferred embodiment of the present invention.

FIGS. 10-12 show a method for utilizing a preferred embodiment of the present invention.

FIG. 13 shows another preferred embodiment of the present invention.

FIG. 14 shows another preferred embodiment of the present invention.

FIGS. 15-16 show a method for utilizing a preferred embodiment of the present invention.

FIG. 17 shows a prior art mayo stand cover covering a mayo stand tray.

FIGS. 18-19 show another preferred embodiment of the present invention.

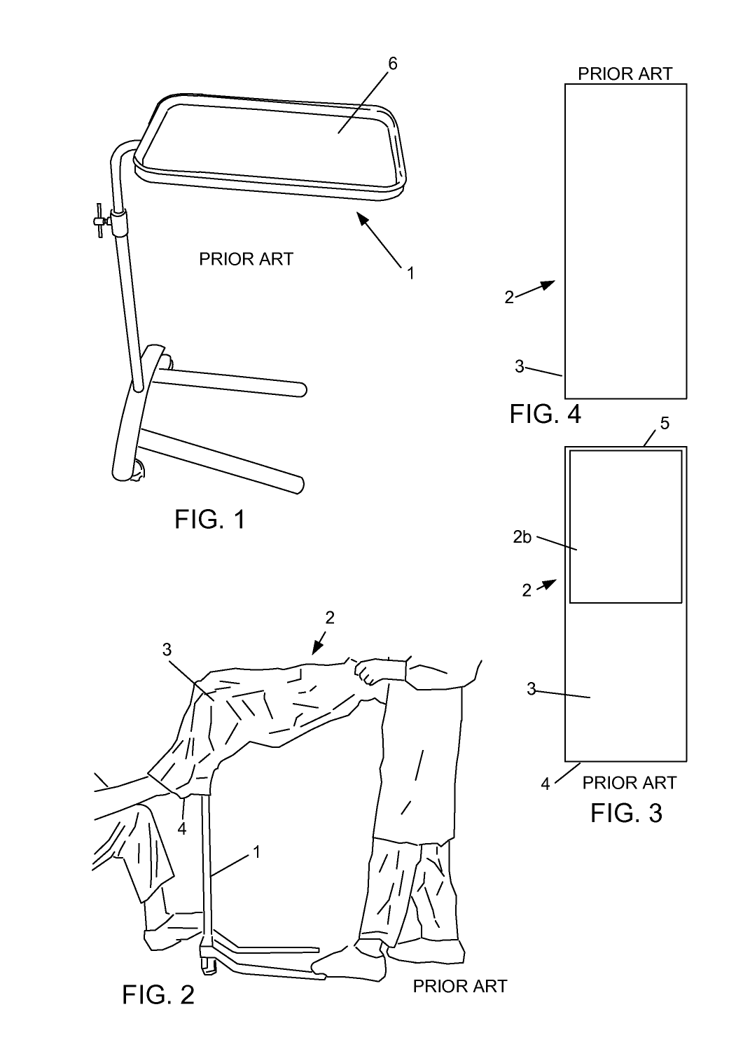

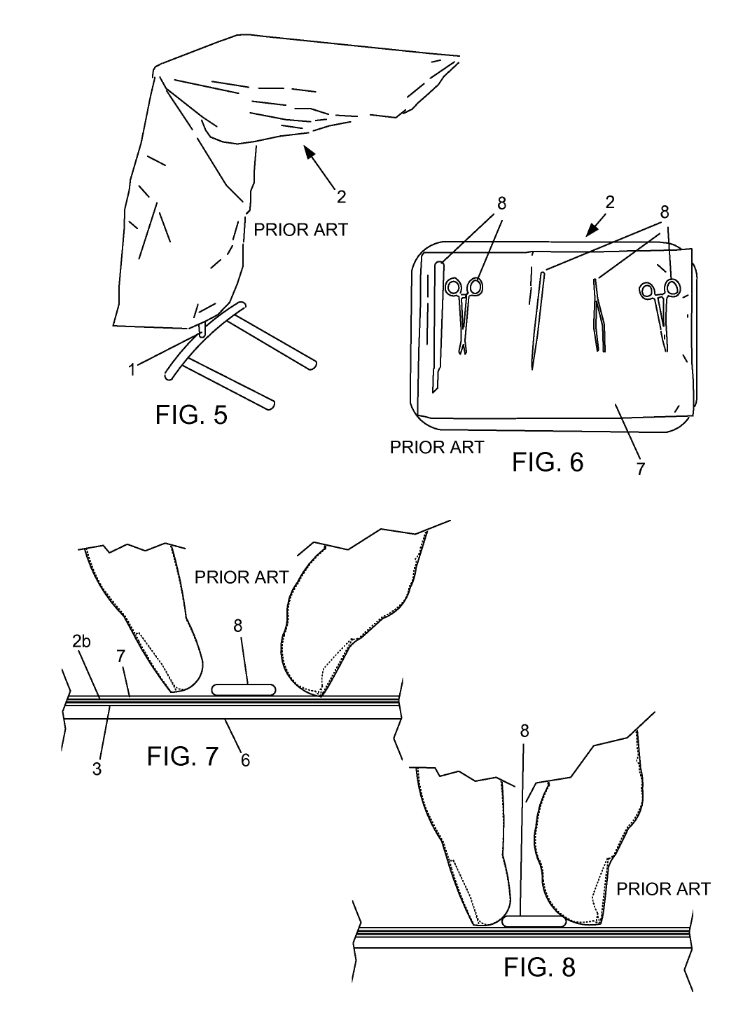

### DETAILED DESCRIPTION OF THE PREFERRED EMBODIMENTS

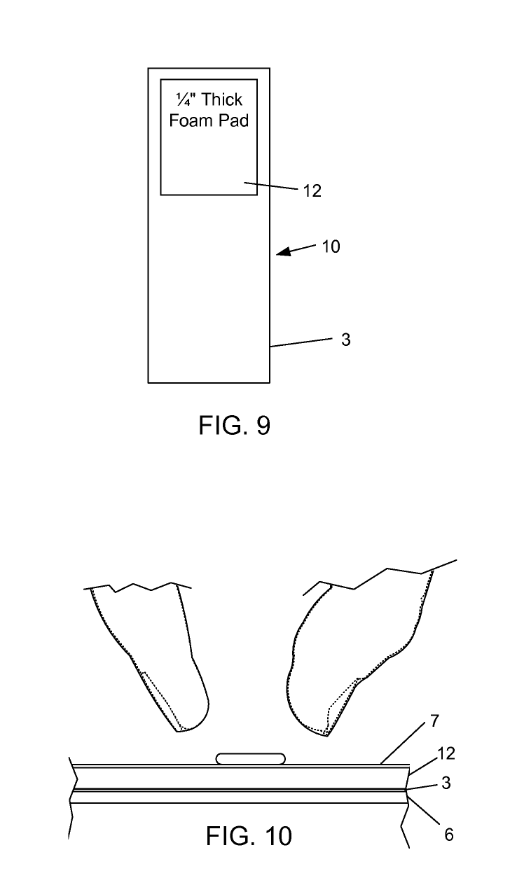

FIGS. 9-12 show mayo cover sheet 10. Mayo cover sheet 10 is preferably a disposable, single use device.

FIG. 9 shows a top view of a preferred embodiment of the present invention. Mayo stand cover 10 includes plastic thin 53 inch×23 inch flexible plastic bag 3 (discussed above in the Background section). Absorbent paper sheet 2b (FIG. 3) has been removed. In its place springy foam pad 12 has been adhesively attached to flexible plastic bag 3 as shown. In a preferred embodiment, springy foam pad 12 is ¼ inch thick soft polyurethane foam. Foam pad 12 is preferably cut into a rectangular sheet of a size that fits inside tray 6 leaving a ½ inch border on each of its 4 sides. For example, tray 6 is approximately 19⅛ inch×12⅝ inch. Therefore, foam pad 12 has been cut into a rectangular shape that is approximately 18⅝ inch×12⅛ inch.

### Utilization of the Preferred Embodiment

FIGS. 10-12 illustrate the utilization of a preferred embodiment of the present invention.

In FIG. 10, the doctor has positioned his hand above tool 8. Tool 8 is resting on cloth 7. Cloth 7 is covering springy foam pad 12. Foam pad 12 covers flexible plastic bag 3 which covers tray 6.

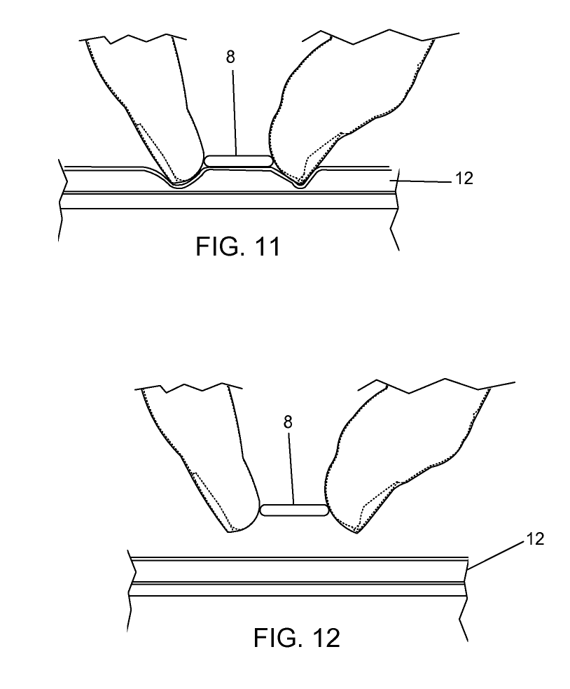

In FIG. 11, the doctor has pressed his finger and thumb down into the areas on each side of tool 8. Springy foam pad 12 is flexible and easily depresses under the force of the doctor's thumb and finger. Spring foam pad 12 is of sufficient softness and thickness so that the doctor can easily press his finger and thumb downward so that he can easily grip the small, flat tool, despite the doctor's large fingers.

In FIG. 12, the doctor has lifted his hand upward and has removed the tool from the top of the cover. Foam pad 12 has sprung back into its original shape. By utilizing foam pad 12, the doctor has been able to easily remove tool 8 from tray 6.

### Other Preferred Embodiments

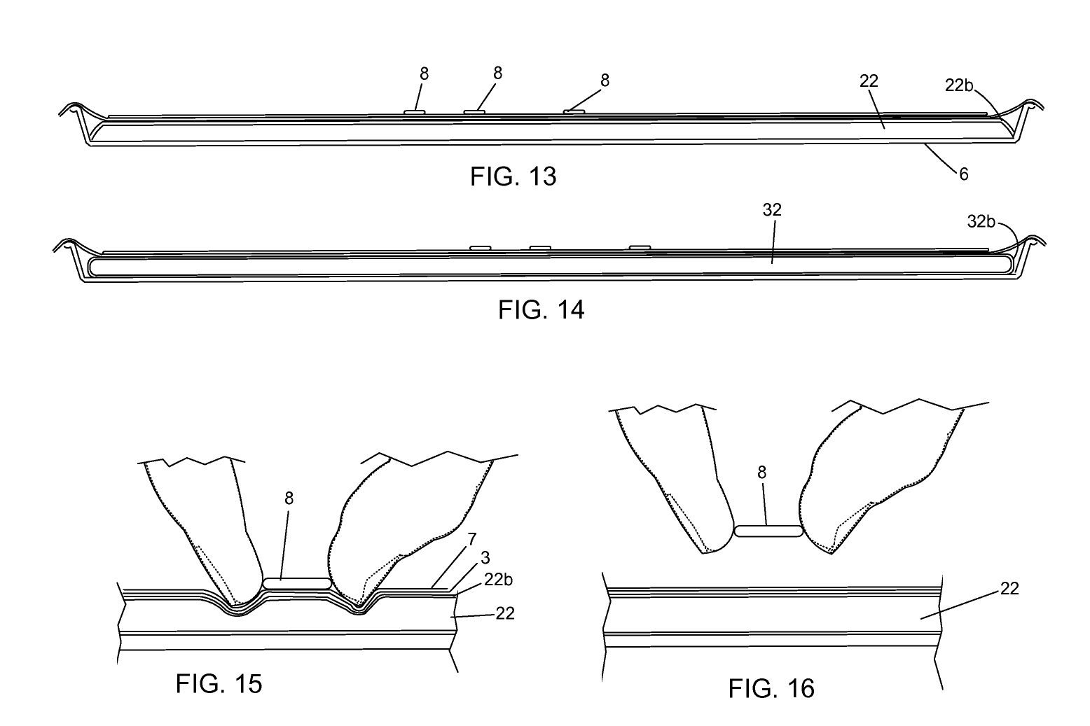

FIG. 13 shows another preferred embodiment of the present invention. FIG. 13 shows a multiple use preferred embodiment. In FIG. 13, foam pad 22 is adhesively attached to tray 6 and is covered by flexible vinyl covering 22b. Flexible bag 3 is draped over foam pad 22 and cloth 7 is laid over flexible bag 3. Surgical tools 8 rest on cloth 7.

FIG. 15 shows the utilization of foam pad 22. In FIG. 15, the doctor has pressed his finger and thumb down into the areas on each side of tool 8. Springy foam pad 22 is flexible and easily depresses under the force of the doctor's thumb and finger. This allows for the doctor to easily grip the small, flat tool, despite the doctor's large fingers.

In FIG. 16, the doctor has lifted his hand upward and has removed the tool from the top of the cover. Foam pad 22 has sprung back into its original shape. By utilizing foam pad 22, the doctor has been able to easily remove tool 8 from tray 6.

After the surgical procedure is over, flexible bag 3 and cloth 7 are removed and discarded. The top of vinyl covering 22b is cleaned and sanitized so that foam pad 22 can be utilized again for subsequent medical procedures.

FIG. 14 shows another preferred embodiment of the present invention. In FIG. 14, foam pad 32 is completely covered by vinyl covering 32b. In the embodiment shown in FIG. 14, foam pad 32 is placed on tray 6. Foam pad 32 can be removed from tray 6 and cleaned and sanitized after usage as appropriate. It can then be placed down on tray 6 again for further usage. It can be flipped over also as appropriate. After multiple usages have occurred and when it is determined that foam pad 32 is no longer cleanable, foam pad 32 can be discarded and replaced.

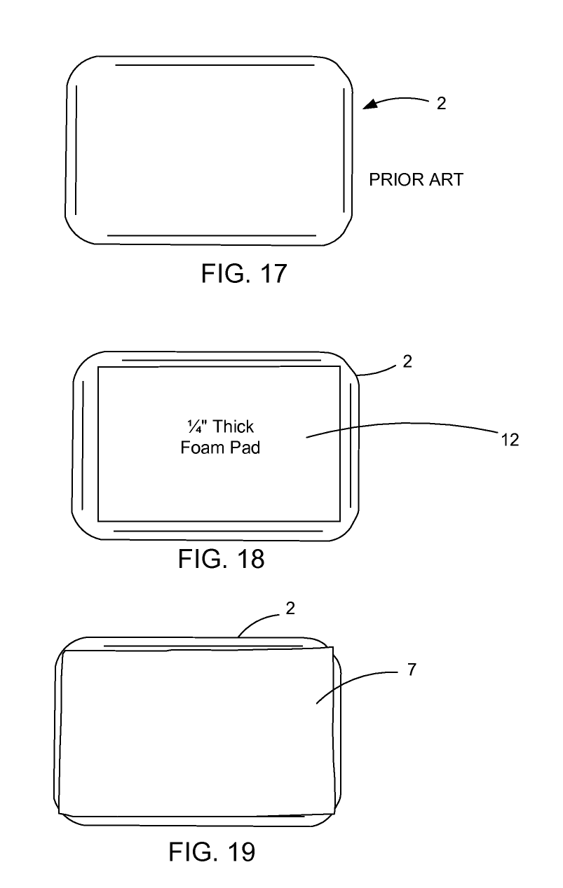

FIGS. 18-19 show another preferred embodiment of the present invention. In FIG. 17, prior art cover 2 is covering a mayo stand tray. In FIG. 18, ¼ inch thick foam pad 12 has been laid down on top of prior art cover 2. In FIG. 19, absorbent cloth 7 has been laid over foam pad 12. The embodiment shown in FIG. 19 functions in a manner similar to that described above in reference to earlier described preferred embodiments.

While the above description contains many specifications, the reader should not construe these as limitations on the scope of the invention, but merely as exemplifications of preferred embodiments thereof. Those skilled in the art will envision many other possible variations are within its scope. For example, although it was stated that springy foam pad 12 is preferably ¼ inch thick soft polyurethane foam, other springy materials could be also be used. For example, springy foam pad 12 could be fabricated from soft springy ¼ inch thick polyethylene foam. The thickness of foam pad 12 can also be modified. Although the preferred thickness is about ¼ inch, thicker or thinner pads could be used but the thickness should not be less than about ⅛ inch. In another preferred embodiment, foam pad 12 is approximately ½ inch thick. Also, although it was stated that covering 22b and 32b are vinyl, it should be understood that other flexible, water resistant materials can also be used. For example, coverings 22b and 32b could be fabricated from flexible plastic. Accordingly the reader is requested to determine the scope of the invention by the appended claims and their legal equivalents, and not by the examples which have been given.

What is claimed is:

1. A mayo stand for holding at least one surgical tool, said mayo stand comprising:
A) a tray defining a tray surface area, and
B) a springy material having an uncompressed thickness of at least ⅛ inch and a surface area slightly smaller than the tray surface area and adapted to fit on the tray to support surgical tools,
wherein said springy material having sufficient softness and thickness so that said springy material is easily depressed downward by finger and thumb force so that said at least one surgical tool is easily graspable.

2. The mayo stand as in claim 1, further comprising:
A) a plastic bag covering said tray, wherein said springy material is positioned on top of said plastic bag and wherein said springy material is positioned inside said tray, and
B) an absorbent cloth positioned over said springy material, wherein said at least one surgical tool is laid on top of said absorbent cloth.

3. The mayo stand as in claim 1, wherein said springy material is a soft foam pad.

4. The mayo stand as in claim 3, wherein said soft foam pad is a polyurethane foam pad.

5. The mayo stand as in claim 3, wherein said soft foam pad is a polyethylene foam pad.

6. The mayo stand as in claim 2, wherein said plastic bag is a single use disposable plastic bag.

7. The mayo stand as in claim 1, wherein said springy material is attached to said said tray, wherein said springy material comprises a water resistant covering for covering the top of said springy material.

8. The mayo stand as in claim 7, further comprising
A) a flexible plastic bag covering said tray and covering said springy material, and
B) a cloth positioned in said tray over said plastic bag and over said springy material, wherein said at least one surgical tool is laid on said cloth.

9. The mayo stand as in claim 1, wherein said springy material is surrounded by a water resistant covering, wherein said springy material is laid inside said tray.

10. The mayo stand as in claim 9, further comprising
A) a flexible plastic bag covering said tray and covering said springy material, and
B) a cloth positioned in said tray over said plastic bag and over said springy material, wherein said at least one surgical tool is laid on said cloth.

11. A mayo stand for holding at least one surgical tool, said mayo stand comprising:
A) a tray, and
B) a springy material positioned on said tray, wherein said surgical tools are laid on top of said springy material,
wherein said springy material is of sufficient softness and thickness so that said springy material is easily depressed downward by finger and thumb force so that said at least one surgical tool is easily graspable.

12. The mayo stand as in claim 11, further comprising:
A) a plastic bag covering said tray, wherein said springy material is positioned on top of said plastic bag and wherein said springy material is positioned inside said tray, and
B) an absorbent cloth positioned over said springy material, wherein said at least one surgical tool is laid on top of said absorbent cloth.
